(19) United States
(12) Patent Application Publication
(10) Pub. No.: US 2026/0232173 A1
(43) Pub. Date: Aug. 13, 2026

(54) REFLECTIVE STICKER FOR DENTAL USE

(71) Applicant: Daniel TSIMERMAN, Vancouver (CA)

(72) Inventor: Daniel TSIMERMAN, Vancouver (CA)

(21) Appl. No.: 19/470,943

(22) PCT Filed: Oct. 25, 2023
(86) PCT No.: PCT/CA2023/051420

§ 371 Date: Sep. 30, 2025

Related U.S. Application Data
(63) Continuation-in-part of application No. 18/300,135, filed on Apr. 13, 2023.

(51) Int. Cl. A61B 1/00; A61B 1/253

(52) U.S. Cl. CPC A61B 1/00096; A61B 1/253

(57) ABSTRACT

Reflective sticker to be applied to a dental instrument. One side of the sticker is reflective, and an opposite side comprises an adhesive that secures the sticker to a receiving surface of the dental instrument. The receiving surface may have any size or shape, and a corresponding reflective sticker may be applied. Similarly, the receiving surface may have any profile, such as generally planar, concave, or convex. The reflective sticker may be flexible and flex to conform to the receiving surface. The adhesive has physical properties, such as thermal resistance properties, such that the reflective sticker is secured to the dental instrument after application and remains attached in normal use, but degrades after an autoclaving process. The adhesive is preferably selected such that the reflective sticker cannot be reused or reattached after autoclaving. In some embodiments, the reflective sticker fully separates from the instrument during the autoclaving process itself.

### TECHNICAL FIELD

The present invention relates to dental mirrors. More specifically, the present invention relates to single-use disposable reflective stickers for application to dental instruments.

### BACKGROUND

When performing dental procedures, a dentist's view of a patient's oral cavity is often obstructed. To allow a dentist a more complete view of the patient's oral cavity, the dentist will often use a dental mirror tool/instrument. The mirror on the tool allows the dentist to observe features that are otherwise concealed. The dental mirror is typically mounted on a handle and the plane of the mirror is at an angle to the handle.

As should be understood, dental mirrors are easily marred, damaged, or require significant cleaning, often almost immediately after the first use, either during dental procedures or during processing in ultrasonic baths, instrument washers, autoclaves, or general handling within the dentist's office. Sterilization of such tools may remove bacteria and debris but cannot repair scratches on the reflective surface itself. Further, seams in conventional tools (such as seams where a conventional mirror portion is joined to a conventional head of a tool) frequently trap debris and microorganisms.

Although dental mirrors are meant to be disposed of after a certain number of uses, they tend to be reused significantly longer than is preferred, largely due to their cost. This represents a problem for both the dentist and patient in that, when a dentist is using a scratched mirror, their view of the treatment area is impaired. This can result in errors in treatment, pain or discomfort for the patient, and/or longer or more difficult dental procedures.

Single-use disposable plastic mirror instruments have been previously introduced, in an attempt to address both the scratch problem and to remove the time required to sterilize regular mirrors. Although such disposable instruments are significantly lower cost than regular dental mirrors, they introduced new concerns, as follows:
(1) Additional storage space is required to accommodate large packages of these instruments, as they each comprise a handle and a mirror portion.
(2) Relatedly, such single-use disposable instruments have a large waste footprint.
(3) The reflective surfaces of such disposable instruments are frequently significantly inferior to standard dental mirrors in terms of reflectivity.
(4) For cost-reduction purposes, such disposable instruments are typically made of low-quality plastic, which feels different for the operator than conventional metal mirrors, and also has a lower-quality appearance.

Additionally, conventional dental mirrors fog easily when used intra-orally (i.e., due to the heat and humidity of the patient's mouth). To remove the fog, dentists commonly wipe the mirror against the inner lining of the patient's cheek. As a result of such wiping motions and of contact with the patient's soft tissue, the mirror surface is frequently covered with various particulate matter, debris, etc., which cannot be removed by the same wiping process. Thus, dental professionals must frequently wipe down the mirror surface (e.g., with a piece of gauze saturated in alcohol). Various coatings have been proposed to reduce the fogging tendency of conventional mirrors (which are conventionally made of rhodium-coated glass). However, adding such coatings to conventional mirrors only increases the cost, which, as described above, frequently leads dentists to use dental mirrors long after they should be disposed of.

Clearly, there is a need for highly reflective and high-quality dental mirrors that can be disposed of without significant waste and replaced at low cost.

### SUMMARY

The present invention provides a reflective sticker to be applied to a dental instrument. One side of the sticker is reflective, and an opposite side comprises an adhesive that secures the sticker to a receiving surface of the dental instrument. The receiving surface may have any size or shape, and a corresponding reflective sticker may be applied. Similarly, the receiving surface may have any profile, such as generally planar, concave, or convex. The adhesive has physical properties, such as thermal resistance properties, such that the reflective sticker is secured to the dental instrument after application and remains attached in normal use, but degrades after an autoclaving process. The adhesive is preferably selected such that the reflective sticker cannot be reused or reattached after autoclaving. In some embodiments, the reflective sticker fully separates from the instrument during the autoclaving process itself.

In a first aspect, this document discloses a reflective sticker, said reflective sticker comprising: a reflective side; and an adhesive side opposite said reflective side, wherein said reflective side is usable as a mirror, wherein said reflective sticker is sized for application to a receiving surface of a dental instrument, and wherein said adhesive side comprises an adhesive that secures said reflective sticker to said receiving surface when said reflective sticker is applied to said dental instrument.

In one embodiment, this document discloses a reflective sticker wherein said adhesive has properties such that said adhesive degrades during autoclaving of said dental instrument.

In one embodiment, this document discloses a reflective sticker wherein the reflective sticker separates from said receiving surface following autoclaving of said dental instrument.

In one embodiment, this document discloses a reflective sticker wherein said properties comprise a low thermal resistance.

In one embodiment, this document discloses a reflective sticker wherein said reflective side has a reflectivity in the visible range greater than about 85%.

In one embodiment, this document discloses a reflective sticker wherein said reflective side comprises a polymer layer.

In one embodiment, this document discloses a reflective sticker comprises a metallic layer.

In one embodiment, this document discloses a reflective sticker further comprising a tongue extending from an application layer atop said reflective side, wherein said tongue is configured for use in applying said mirror to said dental instrument and wherein said tongue is usable for removing said application layer.

In one embodiment, this document discloses a reflective sticker wherein said reflective sticker is applied to a receiving surface that is a generally planar surface.

In one embodiment, this document discloses a reflective sticker wherein said reflective sticker is flexible and said reflective sticker is applied to a receiving surface that is a concave surface.

In a second aspect, this document discloses a sheet of reflective stickers, said sheet comprising: a backing; and a plurality of reflective stickers removably attached to said backing, wherein each of said plurality of reflective stickers comprises a reflective side and an adhesive side opposite said reflective side, wherein said reflective side is usable as a mirror, wherein each of said plurality of reflective stickers is sized for application to a receiving surface of a dental instrument, and wherein said adhesive side comprises an adhesive that secures said reflective sticker to said receiving surface when said reflective sticker is applied to said dental instrument.

In one embodiment, this document discloses a sheet wherein said adhesive has properties such that said adhesive degrades during autoclaving of said dental instrument.

In one embodiment, this document discloses a sheet wherein said adhesive has properties such that said reflective sticker separates from said receiving surface following autoclaving of said dental instrument.

In one embodiment, this document discloses a sheet wherein said properties comprise a low thermal resistance.

In one embodiment, this document discloses a sheet wherein, for each of said plurality of reflective stickers, said reflective layer has a reflectivity in the visible range greater than about 85%.

In one embodiment, this document discloses a sheet wherein, for each of said plurality of reflective stickers, said reflective side comprises a polymer layer.

In one embodiment, this document discloses a sheet wherein, for each of said plurality of reflective stickers, said reflective side comprises a metallic layer.

In one embodiment, this document discloses a sheet wherein each of said plurality of reflective stickers further comprises a tongue extending from an application layer atop said reflective side, wherein said tongue is configured for use in applying said mirror to said dental instrument and wherein said tongue is usable for removing said application layer.

In one embodiment, this document discloses wherein at least one of said plurality of reflective stickers is applied to a receiving surface that is a generally planar surface.

In one embodiment, this document discloses a sheet wherein at least one of said plurality of reflective stickers is flexible and said reflective sticker is applied to a receiving surface that is a concave surface.

### BRIEF DESCRIPTION OF THE DRAWINGS

The present invention will now be described by reference to the following figures, in which identical reference numerals refer to identical elements and in which:

FIG. 1 is a view of a detachable reflective sticker according to an aspect of the present invention;

FIG. 2A is a view of a reflective sticker applied to a dental instrument;

FIG. 2B is a view of a reflective sticker in the process of being applied to a dental instrument;

FIG. 3A is a view of a dental instrument having a receiving surface that is a concave surface;

FIG. 3B is a side view of the dental instrument of FIG. 3A;

FIG. 4 is a schematic side view of a reflective sticker, showing a possible construction;

FIG. 5A is a view of a sheet of reflective stickers according to an aspect of the invention;

FIG. 5B is a view of another sheet of reflective stickers according to an aspect of the invention;

FIG. 5C is a view of another sheet of reflective stickers according to an aspect of the invention; and

FIGS. 6A to 6C are images of exemplary packaging for sheets of reflective stickers.

### DETAILED DESCRIPTION

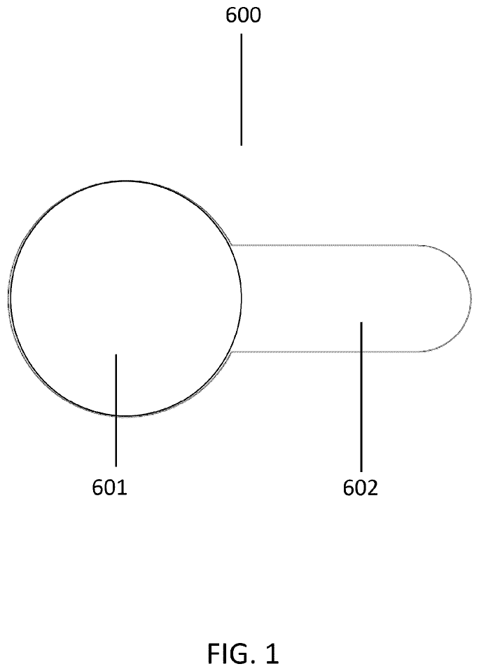

Referring now to FIG. 1, a detachable sticker 600 is shown, which comprises another aspect of the invention. The detachable sticker 600 comprises a mirror portion 601 with a reflective surface/reflective side. The mirror portion 601 covers a receiving surface of a dental instrument when the dental instrument is in use.

The other surface of the detachable sticker 600 is coated with a removable adhesive, allowing the mirror portion 601 to adhere to the receiving surface with adequate strength to remain on the receiving surface during the procedure, yet flexible enough to be easily removed from the receiving surface following the procedure. Preferably, the adhesive releases under the high heat, steam, and pressure of an autoclave, allowing for the easy removal of the detachable sticker 600.

The reflective surface is reflective such that the mirror portion/reflective side 601 reflects a view of the oral cavity towards the dentist when the dental instrument is in use. Preferably, the reflective surface/side is formed of a thin film (e.g., polyethylene terephthalate, “PET”) or sheet coated with a dielectric coating that has a substantially higher reflectivity than a standard rhodium-coated dental mirror. Preferably, the dielectric coating reflects at least 60%, more preferably at least 85%, or even more preferably at least 95%, of visible light.

In a preferred embodiment, the detachable sticker 600 has a tongue 602. The tongue projects radially outwards from the edge of the mirror portion 601. The tongue 602 allows the user to grip the detachable sticker 600 without touching the reflective surface or the adhesive surface. The tongue 602 also helps the user to properly align the mirror portion 601 on the receiving surface of a dental instrument. The tongue 602 can be separated from the mirror portion 601 before the dental mirror tool 100 is used with a patient.

In a further preferred embodiment, the adhesive surface on the mirror portion 601 of the detachable sticker 600 is covered by a disposable protective layer/application layer. The reflective surface of the mirror portion 601 is covered by a disposable film layer that also forms the tongue 602. In such an embodiment, a user removes the disposable protective layer. The user then positions the mirror portion 601 to be centred over a receiving surface of a dental instrument using the tongue 602. The user then affixes the mirror portion 601 to the receiving surface and removes the disposable film layer by lifting the tongue 602. In some embodiments, further, the tongue 602 may be an additional piece of film or material that is attached to the disposable film layer/application layer, rather than integrally formed with the disposable film layer/application layer.

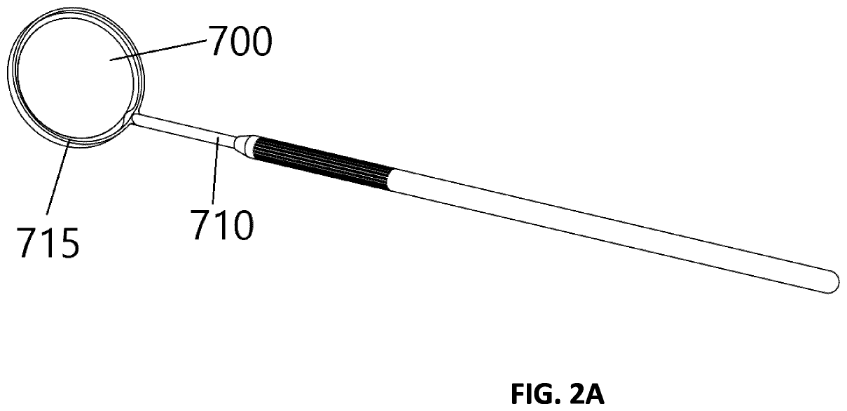

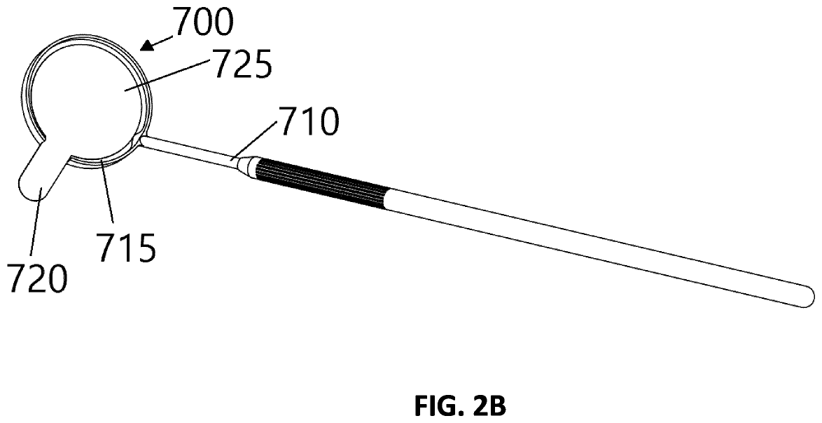

Of course, such reflective stickers may be sized for application to many different dental tools and dental instruments, including non-self-cleaning dental instruments. In one embodiment, shown in FIG. 2A, a reflective sticker 700 is applied to a dental instrument 710. The dental instrument 710 may be any suitable instrument. In typical embodiments, the dental instrument comprises a handle and a receiving surface to which the reflective sticker 700 is sized. As shown in FIG. 2B, the reflective sticker 700 may comprise a tongue 720 (similar to the tongue 602 described above) that is attached to or part of an application layer 725 (e.g., the disposable film layer/disposable protective layer described above). The application layer 725 is a layer atop the reflective surface/reflective side of the reflective sticker 700. The application layer 725 protects the reflective surface/reflective side before the reflective sticker 700 is applied to the receiving surface 715 of the dental instrument 710. As described above, the user may remove the application layer 725 by lifting or pulling on the tongue 720.

Note that, although the exemplary reflective stickers/receiving surfaces shown in these figures are circular, reflective stickers according to the invention may have any suitable shape. Further, any suitably sized or shaped receiving surface may receive a corresponding reflective sticker. As well, the receiving surface may have any suitable curve or curved profile (e.g., concave, convex, planar, variable, etc). Similarly, the receiving surface may also be stationary or may be a moving surface. As an example, the receiving surface may be a spinning or rotating surface, such as, for example, a rotating surface on a dental instrument.

Curved mirrors possess properties which are highly desirable in dentistry. In particular, concave mirrors focus light, using a curved mirror (e.g., a reflective sticker applied to a receiving surface that is a concave surface or a convex surface) significantly increases the light reflection within the oral cavity. Further, curved mirrors create magnification effects. Thus, the use of curved mirrors by a dental professional can increase both intra-oral visibility and detail. As such, curved mirrors, used in addition to conventional light sources such as overhead dental unit lights, light sources mounted on magnifying loupes, additional lighting from an operating microscope, and/or lighting incorporated into a dental handpiece, provide a significant boost in lighting. It has been observed in a clinical setting that a range of focal lengths of concave mirrors from 25 mm to 125 mm provides significant benefits in the oral cavity, in terms of both magnification and illumination. This increase in intra-oral lighting from concave mirrors can be observed even in comparison with dental microscopes (used in the absence of concave mirrors). Dental microscopes employ lighting systems costing tens of thousands of dollars and are effective in illuminating a direct view, but still rely on dentist's handheld mirrors for a reflected view when the dentist cannot see an object directly. As such, cost-effective curved mirrors can provide a significant advantage even over far more costly tools.

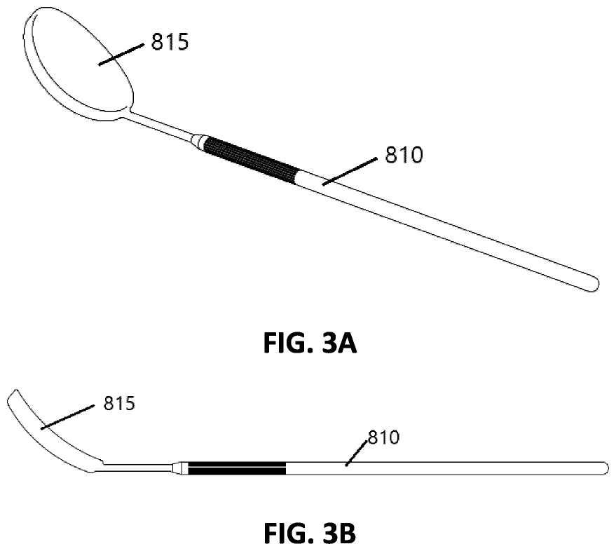

As one example, shown in FIGS. 3A and 3B, the receiving surface 815 of a dental instrument 810 may be a concave surface. In other embodiments, the receiving surface is a convex surface. In other embodiments, the receiving surface is generally planar. Different advantages of different profiles would be apparent to the person of skill in the art, and different appropriate uses of such tools would similarly be apparent.

Note that the reflective sticker is, in some embodiments, made of a flexible material. In such embodiments, a single reflective sticker could flex to adhere to receiving surfaces of different profiles. For example, a suitably flexible reflective sticker could be applied to a planar receiving surface, a concave receiving surface, or a convex receiving surface. In other embodiments, a specifically shaped reflective sticker may be required for use on a specific dental instrument. Note as well that, while higher levels of illumination are almost always desirable, the magnification provided by a concave mirror comes with trade-offs (namely, the greater the magnification, the lower the focus distance). As such, a dentist may use several mirror tools of different curvatures to examine different intra-oral features. Thus, a reflective sticker that is flexible enough to be evenly and smoothly applied to mirror tools of different curvatures may be preferred.

As well, it is possible to apply reflective stickers to receiving surfaces that do not perfectly correspond to the stickers. For example, a circular reflective sticker may be applied to a circular receiving surface having a larger diameter than the diameter of the sticker. Similarly, stickers may be applied to receiving surfaces of different shapes. However, as would be understood by the person of skill in the art, the reflective sticker is preferably applied in such a way that there are no bends, bubbles, cracks, or other flaws in the reflective surface. As such, not all reflective stickers may be suited for application to all possible receiving surfaces. Various parameters of the reflective sticker may influence the selection of an appropriate material: for example, the thickness of the reflective sticker would affect its flexibility (i.e., as thickness increases, flexibility decreases). Thicker and less-flexible reflective stickers may be preferred in some embodiments, such as without limitation rotating-mirror embodiments or where stronger and more granular adhesives are used. In such cases, the thickness of the reflective sticker could mask the bumpy effect of a highly granular adhesive. However, as described above, more-flexible (i.e., thinner) reflective stickers may be preferred in embodiments where the receiving surface is not planar. The person skilled in the art would understand how to balance these considerations for any desired implementation.

As noted above, the adhesive side of the reflective sticker comprises an adhesive that is securely applied and/or bonded to the opposite side of the sticker from the reflective side. In some embodiments, the adhesive side and the reflective side each comprise a separate layer (e.g., a reflective film layer and an adhesive film layer), and the two layers are securely bonded together. In other embodiments, the adhesive side comprises an adhesive applied to one side of a reflective film or other reflective layer. The adhesive has physical properties such that the reflective sticker is securely held to the receiving surface of the dental instrument when applied, but is separable from the dental instrument. Further, the selected adhesive has physical properties such that the adhesive degrades/weakens after an autoclaving process (e.g., autoclaving at 135° C. for about 3 minutes to about 30 minutes, or other standard autoclaving settings). In some embodiments, the reflective sticker completely separates from the dental instrument during autoclaving, such that the reflective sticker cannot be reused. In other embodiments, the separation of the reflective sticker from the dental instrument during the autoclaving process is incomplete but is sufficient to prevent reuse of the reflective sticker. Thus, after autoclaving, a fresh reflective sticker must be applied to the dental instrument. In other embodiments, the reflective sticker does not separate from the dental instrument during a single autoclaving process, but the adhesive is degraded or weakened during autoclaving, such that repeated autoclaving processes are sufficient to separate the reflective sticker from the dental instrument. In some implementations, depending on the autoclaving parameters and the properties of the adhesive, the reflective sticker is fully separated from the dental instrument after two autoclaving sessions, while in other implementations, the reflective sticker may fully separate from the dental instrument after four, five, ten, or more autoclaving sessions. As should be understood, fewer autoclaving sessions before separation is generally preferred, to reduce the use of damaged mirrors.

One adhesive used in exemplary testing has the following properties:

| Property (unit of | Measured according | |
|---|---|---|
| measure) | to standard | Value |
| Adhesion to mirror plate | GB/T2792-1998 | 1.8 |
| (kg/inch) | | |
| Initial tack (3) | GB/T4852-2002 | 2 |
| Holding power | GB/T4851-1998 | 8 |
| (Hr/kg/inch²) | | |
| Tensile strength (mPa) | GB/T13542.2 | MD - 172.04 |
| | | TD - 190.6 |
| Elongation at break (%) | GB/T13542.2 | MD - 107.13 |
| | | TD - 85.17 |
| Thermal shrinkage | GB/T13542.2 | MD - 0.92 |
| (150° C. 30 min) | | TD - −0.07 |
| Temperature range (° C.) | — | −5 to 120 |

However, as should be clear, such properties merely relate to one specific adhesive that is suitable for use in the reflective sticker of the present invention. Although adhesives with the above characteristics are thus suitable for the present reflective sticker, the exact values above are in no way required by the present invention.

In observations and exemplary testing, further, various adhesives were tested. An exemplary adhesive with an adhesive strength/adhesive force of 5.4 N/100 mm was insufficient to secure the reflective sticker to the dental instrument in normal use. As such, higher strength adhesives should be selected. An exemplary adhesive with an adhesive strength/adhesive force of 13 N/100 mm, though secure in normal use, required some effort to remove from the dental instrument (before autoclaving). Accordingly, the adhesive in a preferable embodiment would have an adhesive strength/adhesive force between these values, for example between about 8 N/100 mm and about 13 N/100 mm, or between about 8 N/100 mm and about 11 N/100 mm.

Further, to degrade usefully during autoclaving and/or over several repeated autoclaving sessions, the selected adhesive preferably has a low thermal resistance (i.e., a low upper limit on the temperature range in which it is operable). As should be understood, the operating temperature of the autoclave to be used is preferably significantly higher than the adhesive's upper temperature limit, such that the adhesive requires comparatively few autoclaving sessions before fully degrading/separating and requiring replacement of the reflective sticker. Autoclaving temperatures are commonly about 135° C. As such, an adhesive with an upper temperature limit of under 60° C. or under 70° C. may be used in some embodiments. Of course, the operating parameters (including operating time) of the autoclave are also relevant: an adhesive with a higher upper temperature limit (i.e., close to the standard temperature of the autoclave) may still successfully degrade in an autoclave run for a longer period of time.

However, there may also be trade-offs between low operating temperature ranges and adhesive strength/adhesive force: that is, to ensure the reflective sticker stays attached during normal use may require accepting an adhesive with a higher operating temperature range than would otherwise be preferred. As well, as a further example, one adhesive may be suitable for use with dental instruments made of plastics but not with dental instruments made of steel or other metals (or, of course, vice versa). The person skilled in the art would understand how to balance these factors in any specific implementation.

In some embodiments, the adhesive leaves little to no residue on the receiving surface of the dental instrument. In preferable embodiments, whatever residue remains is easily removable, e.g., with water or alcohol. As should also be clear, the material of the intended dental instrument (e.g., plastic, metal, etc) may affect the removal of any adhesive residue.

In some embodiments, the reflective side of the reflective sticker comprises a polymer-coated layer or a dielectric coated layer. In other embodiments, the reflective side of the reflective sticker comprises a metallic layer, such as an aluminized layer. In still other embodiments, the reflective side comprises an aluminized polymer film, such as aluminized PET. Other metallic layers and coatings may be used, of course, depending on the embodiment.

In some embodiments, the various coatings and/or layers of the reflective side preferably have low surface energies (for example, less than 100 dyn/cm or less than 50 dyn/cm). (Note that rhodium, conventionally used in dental mirrors, has a surface energy of 2.7 J/m² (i.e., 2700 dyn/cm). Other metals commonly also have high surface energies, such as higher than 500 dyn/cm or higher than 1000 dyn/cm.). Materials with low surface energies have low attractive force towards the various particulates within the mouth. As such, a reflective sticker with such a coating can avoid the problems related to mirror fogging described above, while simultaneously being less costly than conventional coated rhodium-glass dental mirrors. That said, in some embodiments, coatings with high surface energies (e.g., metallic coatings or metallicized polymer coatings with surface energies in the hundreds or thousands of Dynes per centimetre) may be preferred: for example, for use when significant water spray is expected during a dental procedure, as the comparatively high surface energy of metallic coatings may encourage water to spread in larger/flatter droplets that result in lower distortion. As well, different coatings provide different reflectivities. For example, aluminum/aluminized coatings have reflectivity of over 85% in the visible range, while silver-based coatings and dielectric coatings provide reflectivity of over 95% in the visible range. Different reflectivity values may suit different needs in different implementations.

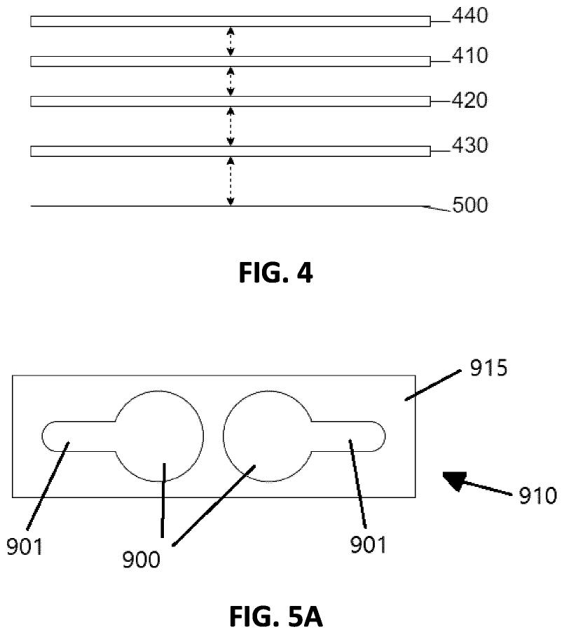

FIG. 4 is a schematic side view of one implementation of a reflective sticker 400. (As should be understood, this figure is not to scale.) In this implementation, the reflective sticker 400 is formed as several layers bonded together. That is, reflective layer 410, such as a reflective film, is bonded or adhered to one side of a substrate 420. An adhesive layer 430, such as an adhesive film, is bonded or adhered to an opposite side of the substrate 420 from the reflective layer 410. The adhesive layer 430 comprises an adhesive, as described above, for adhering the reflective sticker 400 to a receiving surface 500. In some embodiments, an application layer 440 is removably adhered to the reflective layer 410, on an opposite side from the substrate 420. Additionally, in some embodiments, no substrate is used and the reflective layer 410 is directly bonded to the adhesive layer 430.

In some embodiments, the reflective stickers can be provided to dentists individually packed. In such packaging, the reflective stickers may be sterile or non-sterile. In other embodiments, a plurality of reflective stickers can be provided in a single package. In such a single package, the reflective stickers may be non-sterile. In some single-package embodiments, however, the package may be designed so as to preserve sterility of the reflective stickers: for example, the package may comprise numerous individually sealable compartments, for separating individual reflective stickers from each other. As can be imagined, embodiments that reduce both the required storage space and the waste footprint of the reflective stickers are generally preferred.

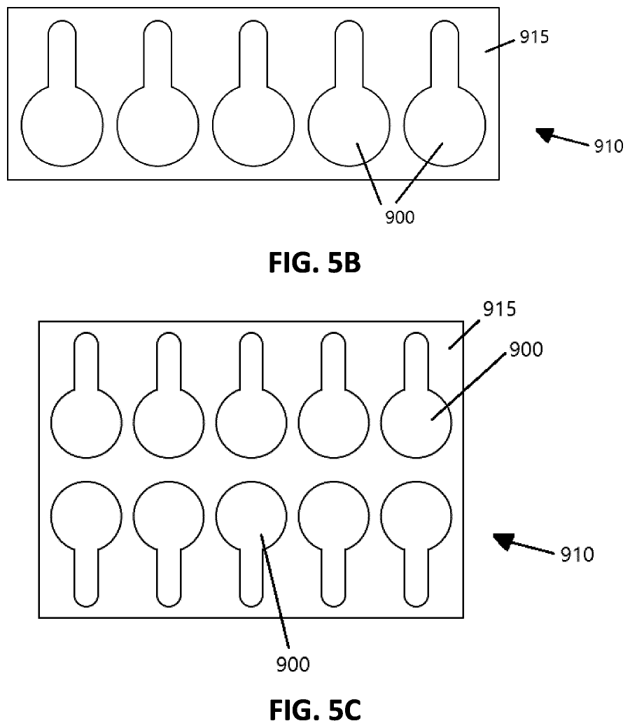

In one aspect, shown in exemplary formats in FIGS. 5A, 5B, and 5C, a sheet 910 of reflective stickers is provided. The sheet 910 comprises a backing 915 and a plurality of reflective stickers 900 removably attached to the backing 915. As would be understood, the adhesive of the reflective stickers 900 is selected so that the reflective stickers 900 may be removed from the backing without interfering with the subsequent adherence of the adhesive to the receiving surface of a dental instrument. The reflective stickers 900, in some embodiments, comprise tongues 901 and/or application layers/protective films atop the reflective side of the reflective sticker. However, as should be understood, embodiments that do not comprise tongues 901 are also possible.

FIG. 5A shows a sheet 910 comprising 2 reflective stickers 900. FIG. 5B shows a sheet 910 comprising 5 reflective stickers 900. FIG. 5C shows a sheet 910 comprising 10 reflective stickers 900. Of course, there is no limit on the number of reflective stickers 900 that may be included on a sheet, depending on the embodiment. These figures merely depict exemplary possibilities but any suitable number of reflective stickers 900 may be provided on the sheet 910.

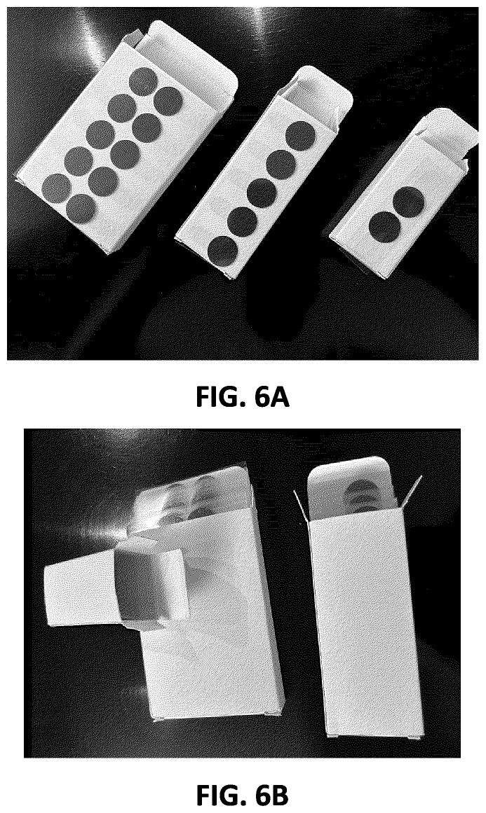

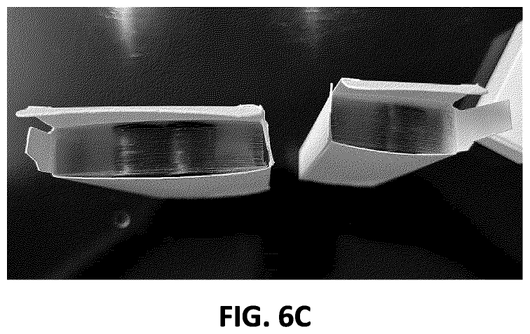

As can also be understood, such sheets require very little storage space. FIGS. 6A, 6B, and 6C show one method of packaging numerous sheets (and, thus, numerous reflective stickers) together, in a compact way. As each reflective sticker shown in these exemplary figures has an associated application layer/protective film, many sheets may be stacked together with little risk to the integrity of the reflective sticker's reflective side. As such, dozens or hundreds of reflective stickers may be packaged together in a compact manner, and each individual reflective sticker has a limited waste footprint, especially as compared to conventional dental mirrors or conventional single-use dental instruments.

Note that, as used herein, the expression ‘at least one of X and Y’ means and should be construed as meaning ‘X, or Y, or both X and Y’. Further, the expression ‘about X’, as used herein and where X is a numerical value, means and should be construed as meaning ‘X plus or minus 10% of X’.

A person understanding this invention may now conceive of alternative structures and embodiments or variations of the above all of which are intended to fall within the scope of the invention as defined in the claims that follow.

We claim:

1. A reflective sticker, said reflective sticker comprising:
a reflective side; and
an adhesive side opposite said reflective side,
wherein said reflective side is usable as a mirror,
wherein said reflective sticker is sized for application to a receiving surface of a dental instrument, and
wherein said adhesive side comprises an adhesive that secures said reflective sticker to said receiving surface when said reflective sticker is applied to said dental instrument.

2. The reflective sticker according to claim 1, wherein said adhesive has properties such that said adhesive degrades during autoclaving of said dental instrument.

3. The reflective sticker according to claim 2, wherein the reflective sticker separates from said receiving surface following autoclaving of said dental instrument.

4. The reflective sticker according to claim 1, wherein said properties comprise a low thermal resistance.

5. The reflective sticker according to claim 1, wherein said reflective side has a reflectivity in the visible range of greater than about 85%.

6. The reflective sticker according to claim 1, wherein said reflective side comprises a polymer layer.

7. The reflective sticker according to claim 1, wherein said reflective side comprises a metallic layer.

8. The reflective sticker according to claim 1, further comprising a tongue extending from an application layer atop said reflective side, wherein said tongue is configured for use in applying said mirror to said dental instrument and wherein said tongue is usable for removing said application layer.

9. The reflective sticker according to claim 1, wherein said reflective sticker is applied to a receiving surface that is a generally planar surface.

10. The reflective sticker according to claim 1, wherein said reflective sticker is flexible and said reflective sticker is applied to a receiving surface that is a concave surface.

11. A sheet of reflective stickers, said sheet comprising:
a backing; and
a plurality of reflective stickers removably attached to said backing,
wherein each of said plurality of reflective stickers comprises a reflective side and an adhesive side opposite said reflective side,
wherein said reflective side is usable as a mirror,
wherein each of said plurality of reflective stickers is sized for application to a receiving surface of a dental instrument, and
wherein said adhesive side comprises an adhesive that secures said reflective sticker to said receiving surface when said reflective sticker is applied to said dental instrument.

12. The sheet according to claim 11, wherein said adhesive has properties such that said adhesive degrades during autoclaving of said dental instrument.

13. The sheet according to claim 12, wherein said adhesive has properties such that said reflective sticker separates from said receiving surface following autoclaving of said dental instrument.

14. The sheet according to claim 11, wherein said properties comprise a low thermal resistance.

15. The sheet according to claim 11, wherein, for each of said plurality of reflective stickers, said reflective side has a reflectivity in the visible range of greater than about 85%.

16. The sheet according to claim 11, wherein, for each of said plurality of reflective stickers, said reflective side comprises a polymer layer.

17. The sheet according to claim 11, wherein, for each of said plurality of reflective stickers, said reflective side comprises a metallic layer.

18. The sheet according to claim 11, wherein each of said plurality of reflective stickers further comprises a tongue extending from an application layer atop said reflective side, wherein said tongue is configured for use in applying said mirror to said dental instrument and wherein said tongue is usable for removing said application layer.

19. The sheet according to claim 11, wherein at least one of said plurality of reflective stickers is applied to a receiving surface that is a generally planar surface.

20. The sheet according to claim 11, wherein at least one of said plurality of reflective stickers is flexible and said reflective sticker is applied to a receiving surface that is a concave surface.
